(19) United States
(12) Patent Application Publication
(10) Pub. No.: US 2003/0158513 A1
(43) Pub. Date: Aug. 21, 2003

(54) Cylindrical assembly for harvesting tissue

(76) Inventors: James Kevin Brannon, Culver City, CA (US); Anthony Charles Griffin, Beverly Hills, CA (US)

Correspondence Address: James K. Brannon, M.D., 5729 Canterbury Drive, Culver City, CA, 90230, US

(21) Appl. No.: 09/973,533

(22) Filed: Oct. 9, 2001

(51) Int. Cl. A61M 1/00

(52) U.S. Cl. 604/35

(57) ABSTRACT

A syringe assembly is constructed for purposes of processing a sample of adipose tissue taken from a human donor desirous of having adipose tissue implanted orthotopically or heterotopically for either cosmetic and therapeutic and reconstructive purposes. A cylindrical body includes an open proximal end and an open conical distal end adapted to fit a dynamic sealing cap or alternately a needle for injecting said adipose tissue. The cylindrical body remains integral and orthogonal in orientation to a suction tube having an aspiration probe used to collect adipose tissue. The syringe assembly further includes a proximal fitting cap having a portal of entry and a portal of exit for purposes of sealing the proximal open end of the cylinder to facilitate the collection of adipose tissue samples. A plunger is provided having operational means for engaging an inner wall of said cylindrical body and axially displacing blood and adipose tissue collected therein. A sterility canister of a size and shade adapted to house the cylinder and prevent contamination of the adipose tissue includes an open proximal end sealable with a containment cap and a closed distal end having a female portion of a size and shape adapted to engage the dynamic sealing cap when the cylinder having been housed therein is centrally disposed to facilitate containment of the blood and adipose tissue during centrifugation.

### BACK GROUND OF THE INVENTION

1. Field of the Invention

Adipose tissue grafting is among one of the most frequently performed surgical procedures by surgeons challenged with reconstructing or aesthetically altering the face or other body parts. Over several years, techniques have been devised to obtain and implant autologous adipose tissue. Scientist and clinicians have sought and defined the essential elements of incorporation of such transplanted tissue and have further desired to secure these elements when considering the benefits of various types of harvesting techniques. The essential elements required for adipose incorporation are viable adipose cells, a healthy host bed, and the absence of infection. Further, clinicians have recognized the consequences of donor site morbidity and prolonged recovery. To circumvent some of these issues, numerous techniques have been devised for fat grafting. The essential element regarding the invention described herein is the ability to harvest adipose tissue samples using a minimally invasive technique. Further, one is able to maintain the sterility of the samples during whole blood separation and preparation for injection into an orthotopic or heterotopic site for therapeutic reconstructive purposes.

2. Information Disclosure Statement

To date, prior art fails to describe a method or apparatus for harvesting an adipose tissue sample that includes a canister orthogonal to a suction tube capable of withstanding a centrifugal force for purposes of separating whole blood from said adipose tissue sample and further using said canister for percutaneous deliver of said adipose tissue sample. Katz, M.D., et. al., in a recent publication of Plastic and Reconstructive Surgery, Vol. 107, No. 2, February 2001, 595-597, describes a plastic bag operationally disposed relative to a suction apparatus for adipose harvesting, but makes no effort to use the same plastic bag to process and deliver the harvested tissue.

### SUMMARY AND OBJECTS OF THE PRESENT INVENTION

It is an object of the present invention to provide a method for percutaneously obtaining a large volume of adipose tissue for fat grafting.

It is yet a further object of the present invention to provide means for processing a volume of harvested adipose tissue.

It is yet a further object of the present invention to ensure that the harvested adipose tissue retains all of the essential elements deemed necessary for rapid incorporation of an autogenous adipose tissue sample.

It is yet a further object of the invention to promote all surgeons concerned with the use of adipose tissue to maintain the sterility of the harvested tissue during centrifugation and preparation for injection.

### SUMMARY

The invention describes a novel and unobvious method for processing harvested adipose tissue. A hollow cylindrical rod is percutaneously inserted into a region of the human body designated as the donor site. A suction tube having a cylindrical canister orthogonal thereto is integral with the hollow cylindrical rod. The suction tube draws adipose tissue samples into the cylindrical canister. After a sufficient quantity of adipose tissue is obtained, the cylindrical canister is disengaged from the suction tube to allow a sealing cap to be engaged thereon. The open conical distal end of the cylindrical canister is closed with a dynamic sealing cap that is coaxial with a female portion in a sterility canister such that during centrifugation, the sealing cap is dynamically sealed so as to avoid extrusion of the blood and adipose tissue sample contained therein the cylindrical canister. After centrifugation, the cylindrical assembly is aseptically removed from the sterility canister and a piston having a plunger attached thereto is operationally disposed therein the open proximal end of the cylindrical canister to forcibly and sequentially deliver the blood and adipose tissue through the open conical end.

### BRIEF DESCRIPTION OF THE DRAWINGS

FIG. 1 is a sectional view of the cylinder having a conical distal end with a dynamic sealing cap fitted thereto of the present invention.

FIG. 2 is a sectional view of the cylinder of the present invention as in FIG. 1, but having adipose tissue therein.

FIG. 3a shows the cylinder of the present invention with the proximal fitting cap moved and a proximal closure cap attached thereto in preparation for centrifugation.

FIG. 3b shows the sterility canister of the present invention having a containment cap.

FIG. 3c shows the containment canister having contained the cylinder of the present invention in preparation for centrifugation.

FIG. 3d shows the sterility canister having contained the cylinder during the process of centrifugation.

FIG. 4 shows the cylinder of the present invention after centrifugation.

FIG. 5 shows the cylinder of the present invention with a plunger engaged in the proximal end for purposes of advancing the contents therein distally.

### DESCRIPTION OF THE PREFERRED EMBODIMENTS

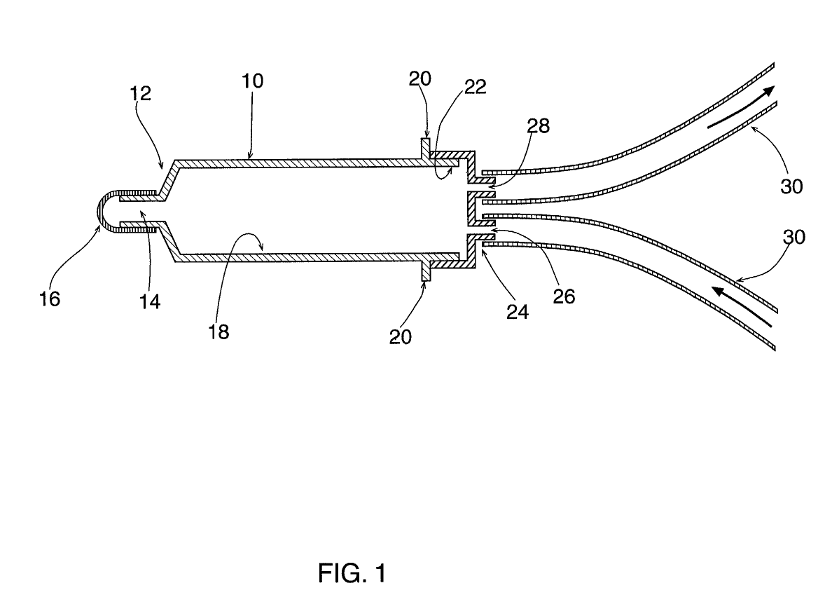

There shown generally at 6 in FIG. 1 is a cylinder 10 having a proximal opening 8 and a partially closed distal end 12 having a distal portion 14 adapted to sealably engage a dynamic sealing cap 16. The cylinder further includes an inner wall 18 extending from the open proximal end 8 to the partially closed distal end 12. A detachable proximal closure cap 24 is adapted to salable engage a cylindrical extension 22 so as to establish a chamber for the collection of adipose tissue. The detachable proximal closure cap 24 has a portal of entry 26 and a portal of exit 28 having been adapted to receive transparent tubing integral with an adipose harvesting probe and a vacuum source.

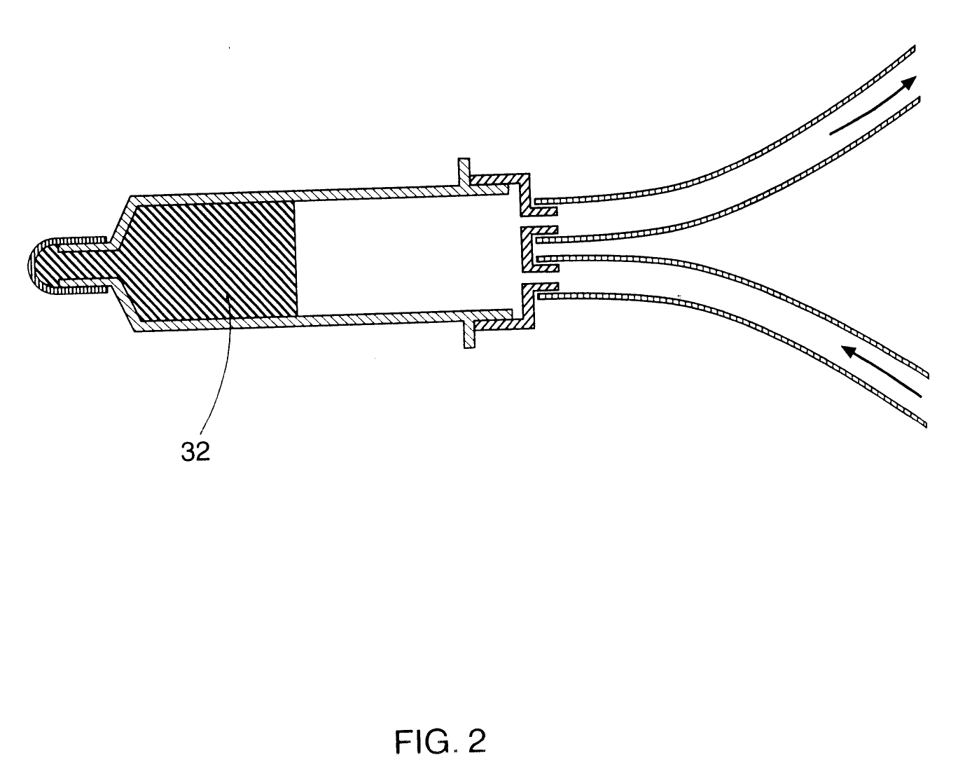

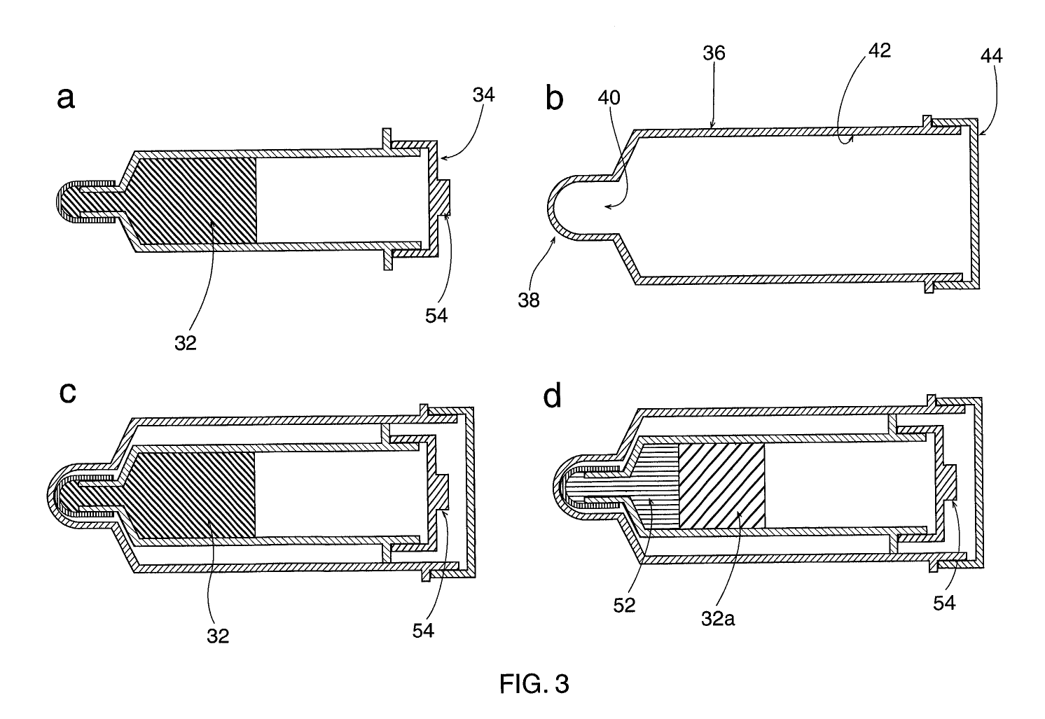

FIG. 2 shows the completed cylindrical assembly with the detachable proximal closure cap 24 having been sealably engaged with the cylindrical extension 22. A plurality of proximal flanges 20 is shown distal to the closure cap 24. Operationally, adipose tissue 32 is readily collected into the cylindrical assembly through the portal of entry 26 as a vacuum source 56 is integral with the portal of exit 28 having transparent tubing 30 attached thereto. FIG. 3a shows the proximal closure cap 24 having been removed and a proximal fitting cap 34 seably engaging the proximal cylindrical extension 22 in preparation for centrifugation. In FIG. 3b, a centrifugation sterility canister 36 is shown having a female portion 40 adapted to receive the dynamic sealing cap 16 in balanced congruity and thereby establish an articulating single first point of balanced fixation at the juncture thereof and said female portion. The proximal flange 20 establishes additional points of fixation along an inner side 42 at a location within the sterility canister 36 so as to establish three-point fixation and ensure balanced centrifugation of the contents therein and induce a blood centrifugate 52 to remain distal to a collection of blood-free adipose tissue 32a after the adipose tissue 32 has been centrifuged. FIG. 3c shows the sterility canister 36 as described having contained the cylindrical assembly 10 and thereby establishing three-points of fixation at the female portion 40 and the plurality of proximal flanges 22. The centrifugation sterility canister is closed with a containment cap 44 and in such configuration the cylindrical assembly having adipose tissue contained therein can now be centrifuged. FIG. 3d shows the cylindrical assembly 10 after centrifugation. The cylindrical assembly is aseptically removed from the sterility canister by using a surgical clamp 56 to deliver the cylindrical assembly therefrom. The adipose tissue 32 having been centrifuged establishes said collection of blood-free adipose tissue 32a and said blood centrifugate 52.

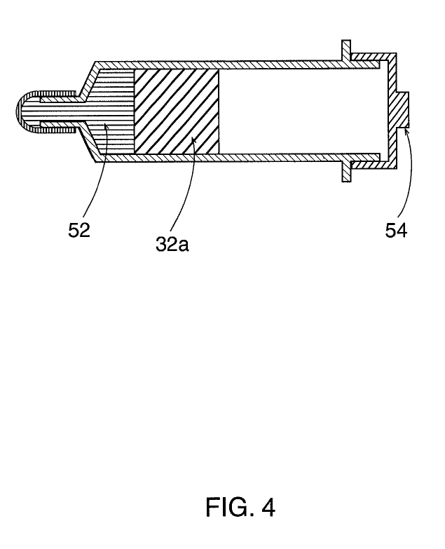

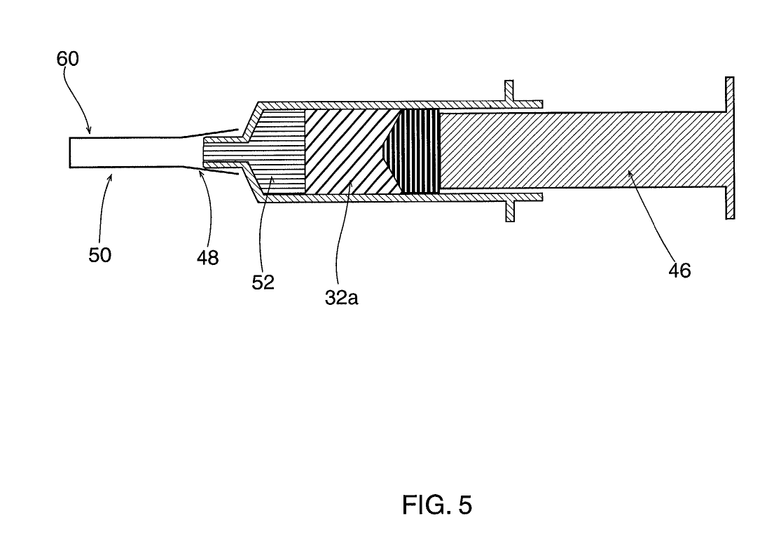

Now shown in FIG. 4 is the cylindrical body 10 with a hypodermic needle 60 having a female hub 48 and a needle portion 50 detachably engaging the male portion 14. A piston rod 46 having been inserted into the proximal portion of the cylindrical body 10 is in operational relationship with the inner wall 18 so as to allow the operator thereof to freely express the centrifuged contents of the cylindrical body in a sequential order.

The invention at hand is a unique way of obtaining a large amount of adipose tissue for grafting purposes while simultaneously maintaining the sterility of the tissue sample, and should not be construed narrowly as modifications do not deviate from the spirit of the invention.

Thus having described the invention at hand, what we desire to claim and secure by Letters Patent is:

1. A device for harvesting tissue comprising:
a cylindrical body having an open proximal end, an inner wall, a partially closed distal end having a male portion of a size and shape adapted to detachably and hermetically receive a dynamic sealing cap, and a proximal closure cap having a portal of entry and a portal of exit so as to establish a chamber for the collection of tissue, said dynamic sealing cap is of a size, shape and dimension adapted to engage a female portion of a centrifugation sterility canister so as to establish an articulating first point of fixation within said sterility canister.

2. A device as defined in claim 1 wherein said cylindrical body further includes a first proximal flange and a second proximal flange, said first and second proximal flanges project outwardly from said cylindrical body so as to articulate along said inner wall and establish additional points of fixation of said cylindrical body within said sterility canister, said first and said additional points of fixation induce a blood centrifugate to remain distal to a collection of blood free adipose tissue

3. A device as defined in claim 1 wherein said cylindrical body further includes a proximal extension, said proximal extension is of a size and shape to detachably and hermetically receive said proximal closure cap.
